(19) United States
(12) Patent Application Publication
(10) Pub. No.: US 2007/0299342 A1
(43) Pub. Date: Dec. 27, 2007

(54) ULTRASOUND DIAGNOSIS APPARATUS AND THE CONTROLLING METHOD THEREOF

(76) Inventor: Kazuyoshi Hayasaka, Tokyo (JP)

Correspondence Address: Patrick W. Rasche;Armstrong Teasdale LLP, Suite 2600, One Metropolitan Square, St. Louis, MO, 63102, US

(21) Appl. No.: 11/765,875

(22) Filed: Jun. 20, 2007

(30) Foreign Application Priority Data
Jun. 23, 2006 (JP) 2006-174471

(51) Int. Cl. A61B 8/14

(52) U.S. Cl. 600/443

(57) ABSTRACT

In order to facilitate the operation and improve the operating efficiency, after a body mark indicating the shape of the imaging region of an examinee is displayed on the display screen, the coordinate positions of the body mark displayed on the display screen are input. The operation of an ultrasound diagnosis apparatus is then controlled based on the input coordinate positions of the body mark.

### CROSS REFERENCE TO RELATED APPLICATIONS

This application claims the benefit of Japanese Application No. 2006-174471 filed Jun. 23, 2006.

### BACKGROUND OF THE INVENTION

The present invention relates to an ultrasound diagnosis apparatus and the controlling method, in particular to an ultrasound diagnosis apparatus which performs a scan of transmitting an ultrasound beam to an imaging region of an examinee and receiving the ultrasound echo reflected in the imaging region to obtain echo signals, to generate ultrasound diagnosis image of the imaging region.

The ultrasound diagnosis apparatus is widely used in the medical field including the fetus examination and heart examination because of the capability of displaying a real-time ultrasound diagnosis image when performing a scan.

When displaying an ultrasound diagnosis image by using an ultrasound diagnosis apparatus, a scan is performed for transmitting an ultrasound beam to the imaging region of an examinee to receive an ultrasound echo reflected in the imaging region, to thereby obtain echo signals. The scan is performed in such scan method as the sector scan method, the linear scan method, the convex scan method, and the radial scan method.

Based on the echo signals obtained by performing the scan, an ultrasound diagnosis image for the imaging region is generated to display on a display screen. In the ultrasound diagnosis apparatus there are many display modes such as A mode, B mode, C mode, and CFM (Color flow mapping) mode, and an ultrasound diagnosis image in correspondence with the mode used is displayed on the display screen.

In this case a body mark is displayed on the display screen indicating the shape of the imaging region of the examinee along with the ultrasound diagnosis image (for example see patent reference 1 and patent reference 2).

[patent reference 1] Japanese Unexamined Patent Publication No. 2005-118

[patent reference 2] Japanese Unexamined Patent Publication No. 2004-57379

For configuring the image quality setting, measuring setting, or comment setting in an ultrasound diagnosis apparatus, the parameters corresponding to the desired setting are selected by selecting an application. The application selection is independent in each setting such as the image quality setting, measuring setting and comment setting. The operation is thereby complex and it is difficult to improve the operation efficiency.

The defect may become apparent when imaging a plurality of imaging regions of the same examinee.

### SUMMARY OF THE INVENTION

It is desirable that the problem described previously is solved.

In one aspect of the invention, the ultrasound diagnosis apparatus for performing a scan by transmitting an ultrasound beam to an imaging region of an examinee and receiving ultrasound echoes reflected in the imaging region, to obtain echo signals for generating an ultrasound diagnosis image of the imaging region, comprising a display unit for displaying on a display screen a body mark indicating the shape of the imaging region, an input unit for inputting the coordinate position of the body mark displayed on the display screen by the display unit, and a control unit for controlling the operation of the ultrasound diagnosis apparatus based on the coordinate position of the body mark input by the input unit.

In another aspect of the invention, the controlling method of an ultrasound diagnosis apparatus for performing a scan by transmitting an ultrasound beam to an imaging region of an examinee and receiving ultrasound echoes reflected in the imaging region, to obtain echo signals for generating an ultrasound diagnosis image of the imaging region, comprising the steps of displaying a body mark indicating the shape of the imaging region on the display screen, inputting the coordinate position of the body mark displayed on the display screen by the display step, and controlling the operation of the ultrasound diagnosis apparatus based on the coordinate position of the body mark input by the input step.

In accordance with the invention, an ultrasound diagnosis apparatus and its controlling method may be provided which allows improvement of the operation efficiency.

Further objects and advantages of the present invention will be apparent from the following description of the preferred embodiments of the invention as illustrated in the accompanying drawings.

### BRIEF DESCRIPTION OF THE DRAWINGS

FIG. 1 shows a schematic block diagram indicative of the arrangement of an ultrasound diagnosis apparatus 1 of the first preferred embodiment in accordance with the invention;

FIG. 2 shows a schematic flow diagram indicative of the operation when performing settings such as image quality setting, measurement setting, and comment setting of the first preferred embodiment in accordance with the invention;

FIGS. 3(a), 3(b), and 3(c) show a schematic diagram indicative of the operation when performing settings such as image quality setting, measurement setting, and comment setting of the first preferred embodiment in accordance with the invention;

FIG. 4 shows a schematic diagram indicative of the input of the coordinate positions of the body mark in the first preferred embodiment in accordance with the invention;

FIG. 5 shows an ultrasound diagnosis image displayed by the operation being set in the first preferred embodiment in accordance with the invention; and

FIGS. 6(a), 6(b), and 6(c) show an ultrasound diagnosis image displayed when performing the measurement setting in the second preferred embodiment in accordance with the invention.

### DETAILED DESCRIPTION OF THE INVENTION

### First Embodiment

(Apparatus)

The ultrasound diagnosis apparatus 1 in accordance with the first preferred embodiment of the invention will be described herein below.

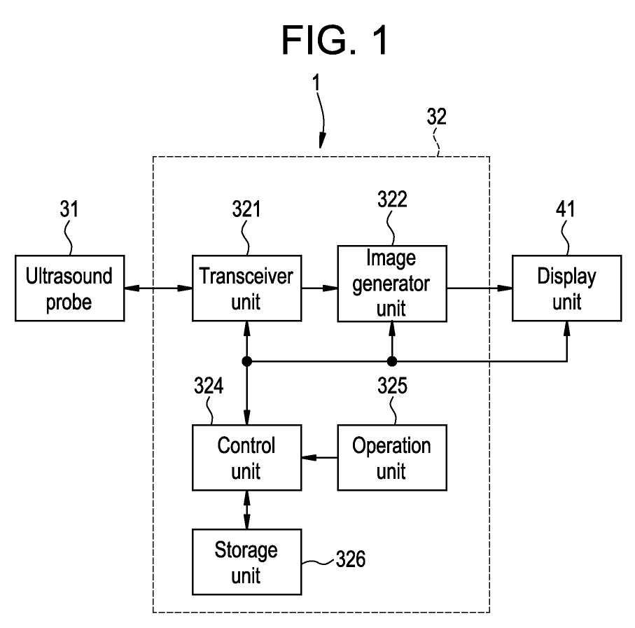

FIG. 1 shows a schematic block diagram indicative of the arrangement of the ultrasound diagnosis apparatus 1 in accordance with the first preferred embodiment of the invention.

As shown in FIG. 1, the ultrasound diagnosis apparatus 1 in accordance with the preferred embodiment of the invention includes an ultrasound probe 31, an operation console 32, and a display unit 41, for performing a scan by transmitting an ultrasound beam to the imaging region of an examinee and receiving ultrasound echoes reflected from the imaging region to obtain the echo signals, thereby generating an ultrasound diagnosis image of the imaging region based on the echo signals. Each component will be described in greater details herein below.

The ultrasound probe 31 may be of the convex type, and has a plurality of ultrasound transducers uniformly arranged (not shown in the figure). The ultrasound transducer used in the ultrasound probe 31 may include piezoelectric materials such as PZT (lead zirconate titanate) ceramics, and transmits ultrasound waves transduced from electric signals and receives ultrasound waves to be transduced to electric signals to output as echo signals. The ultrasound probe 31 in the embodiment is used such that the plane having the ultrasound transducer arranged therein is placed in contact with the surface of the examinee. The ultrasound probe 31 performs a scan by transmitting the ultrasound beam to the body of the examinee in response to the driving signals from a transceiver unit 321 based on the control signal output from a control unit 324 in the operation console 32, and by receiving the ultrasound echoes of the transmitted ultrasound beam reflected from the body of the examinee to obtain the echo signals as raw data. It then outputs the echo signals to the transceiver unit 321.

The operation console 32 has the transceiver unit 321, an image generator unit 322, the control unit 324, an operation unit 325, and a storage unit 326 as shown in FIG. 1. Each component of the operation console 32 includes a data processing unit to conduct data processing.

The transceiver unit 321 includes a transceiver circuit for transmitting and receiving ultrasound waves to the ultrasound probe 31, and directs the ultrasound probe 31 to transmit an ultrasound beam from the ultrasound transducer to the examinee based on the control signal from the control unit 324 and to receive the ultrasound echoes reflected from the examinee by the ultrasound transducer to generate echo signals. For example, the transceiver unit 321 performs a scan of the examinee by the electronic convex scan method so as to obtain echo signals, then outputs the echo signals as acoustic ray data to the image generator unit 322. More specifically, the transceiver unit 321 drives a plurality of ultrasound transducers by switching the position thereof in the ultrasound probe 31 so as to scan the examinee by moving the ultrasound beam, to obtain echo signals. Processing such as amplification, delay, or addition is applied on the echo signals, to output them as the acoustic ray data to the image generator unit 322.

The image generator unit 322, based on the acoustic ray data output from the transceiver unit 321, generates an ultrasound diagnosis image of the imaging region of the examinee. The image generator unit 322 is controlled by the control unit 324 in response to the instruction input to the operation unit 325, in order to generate the ultrasound diagnosis image as B mode image or CFM image. The ultrasound diagnosis image thus generated is temporarily stored in a cine-memory (not shown in the figure) then output to an HDD (not shown in the figure) to be stored.

The control unit 324 includes for example a computer, and a memory for storing a program for executing a predetermined data processing on the computer, supplies control signals to the components based on the operation signals from the operation unit 325 to control the operation of the components. As will be described in greater details later, in this preferred embodiment, the control unit 324 controls the operation of the ultrasound diagnosis apparatus 1 based on the coordinate position of the body mark to be input from the operation unit 325. The control unit 324 will extract some parameters in correspondence with the coordinate position of the body mark to be input from the operation unit 325 from the database in which each of coordinate positions of the body mark is mutually associated by the storage unit 326 with each of the parameters for setting the operation of the ultrasound diagnosis apparatus, then control the operation of the ultrasound diagnosis apparatus so as to correspond to the extracted parameters.

The operation unit 325 may include for example a keyboard (not shown in the figure), and a track ball (not shown in the figure). The operation unit 325 has the operation information input by the operator to output the operation signals to the control unit 324 based on the operation information. As will be described in greater details later, in this preferred embodiment, the operation unit 325 receives the input of the coordinate positions of the body mark displayed on the display screen by the display unit 41. The operation unit 325 may be also constituted by input devices such as a touch panel, a foot switch, and a voice input device.

The storage unit 326 may include for example a memory to store data. In this preferred embodiment, the storage unit 326 stores a database in which each of coordinate positions of the body mark is mutually associated with each of parameters for setting the operation of the ultrasound diagnosis apparatus. More specifically, the storage unit 326 stores a database in which the image quality setting parameters for setting the image quality of the ultrasound diagnosis image are associated as parameters with the coordinate positions of the body mark. The storage unit 326 stores a database in which the measurement setting parameters for setting the measurement of the ultrasound diagnosis image are associated as parameters with the coordinate positions of the body mark. The storage unit 326 also stores a database in which comment setting parameters for setting the comment for the ultrasound diagnosis image are associated as parameters with the coordinate positions of the body mark.

The display unit 41 may include for example an LCD device (not shown in the figure) having a plane display screen and a DSC (digital scan converter) (not shown in the figure), to display the ultrasound diagnosis image generated by the image generator unit 322. As will be described in greater details later, in this preferred embodiment, the display unit 41 displays the body mark indicating the shape of the imaging region to be imaged in the examinee on the display screen.

(Operation)

Now the operation of the ultrasound diagnosis apparatus 1 in accordance with the preferred embodiment of the invention will be described in greater details by way of example of performing settings such as image quality setting, measurement setting, and comment setting.

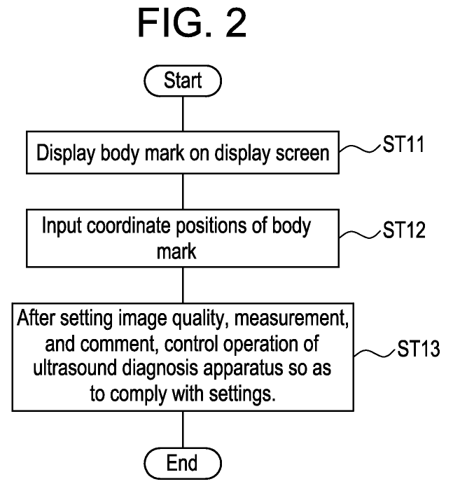

Referring to FIG. 2, there is shown a schematic flow diagram indicative of the operation of the first preferred embodiment of the invention when performing settings such as image quality setting, measurement setting, and comment setting.

First, as shown in FIG. 2, the body mark is displayed on the display screen (ST11).

The display unit 41 displays on the display screen the body mark indicative of the shape of the imaging region to be imaged of the examinee.

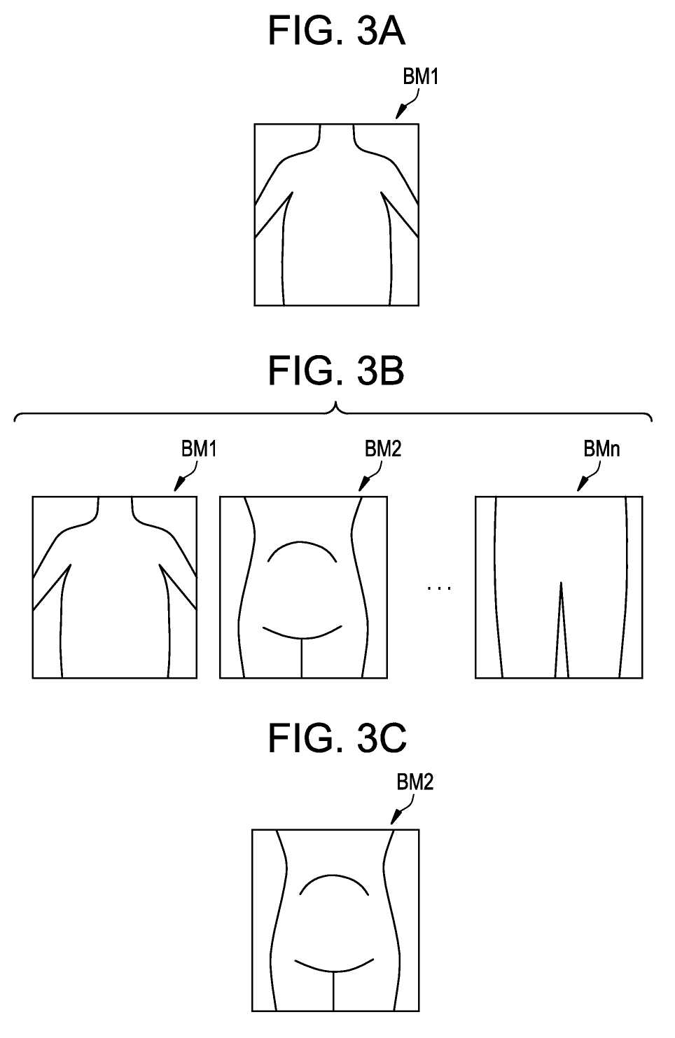

Referring to FIG. 3, there is shown a schematic diagram indicative of the body mark displayed in the first preferred embodiment of the invention.

In this preferred embodiment, in the registration screen for the information of the examinee as the patient or the scan display, the display unit 41 displays on the display screen a first body mark BM1 indicative of the shape of the region corresponding to the area from the shoulder to the abdomen of human body as shown in FIG. 3 (a). To perform imaging of the area outside the imaging area corresponding to the first body mark BM1, a body mark is selected by key input by the operator, from within a plurality of body marks BM1, BM2, . . . , BMn including the first body mark BM1, each assigned to a key on the keyboard of the operation unit 325 as shown in FIG. 3 (b). When the second body mark BM2 indicative of the region corresponding to the lower abdominal region of a pregnant woman as shown in FIG. 3 (b) is selected, the display unit 41 then will display only the second body mark BM2 on the display screen as shown in FIG. 3 (c).

Next, as shown in FIG. 2, the coordinate positions of the body mark are to be input (ST12).

In this example the operator selects the coordinate positions of the body mark displayed on the display screen by the display unit 41 to input to the operation unit 325.

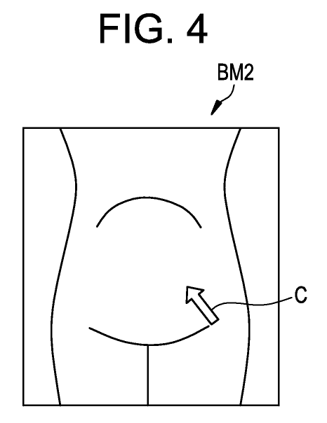

Referring to FIG. 4, there is shown a schematic diagram illustrating the input of coordinate positions of the body mark in the first preferred embodiment in accordance with the invention.

In this preferred embodiment, as shown in FIG. 4, on the display screen displaying the second body mark BM2 indicative of the shape of the region corresponding to the lower abdominal region of a pregnant woman, the operator performs an operation to move the cursor C. Then the operator specifies and inputs the coordinate position in the body mark MB2 by performing the operation of the track ball or the operation of some buttons. For example, as shown in FIG. 4, the operator inputs the coordinate positions in the second body mark BM2, which corresponds to the lower abdominal region containing the fetus.

Next, as shown in FIG. 2, after performing some settings such as image quality setting, the operation of the ultrasound diagnosis apparatus is controlled so as to correspond to the setting (ST13).

In this preferred embodiment the control unit 324 performs the settings based on the coordinate positions of the body mark to be input from the operation unit 325. Thereafter, the operation of the ultrasound diagnosis apparatus will be controlled so as to correspond to the setting. More specifically, the database stored in the storage unit 326 is used in which each of the coordinate positions of the body mark is mutually associated with each of the parameters for setting the operation of the ultrasound diagnosis apparatus. First, from the database stored in the storage unit 326 by associating each of the coordinate positions of the body mark with each of the parameters for setting the operation of the ultrasound diagnosis apparatus, some parameters corresponding to the coordinate position of the body mark to be input by the operation unit 325 will be extracted by the control unit 324. Then the control unit 324 will automatically control the operation of the ultrasound diagnosis apparatus so as to comply with the extracted parameters.

In this preferred embodiment, as have been described above, since the coordinate positions corresponding to the lower abdominal regions containing a fetus in the second body mark BM2 are input, the image quality settings for imaging a fetus are extracted from the database, to automatically perform the setting so as to comply with the extracted parameters. In other words, the image quality setting parameters are automatically set by using the database in which the image quality parameters for setting the image quality of the ultrasound diagnosis image are associated with the coordinate positions of the body mark. Then the control unit 324 will automatically control the operation of the ultrasound diagnosis apparatus 1 so as to comply with the settings.

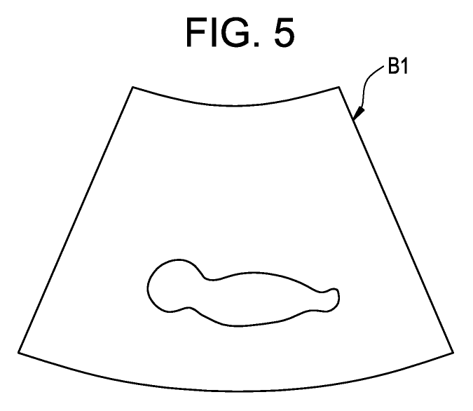

Referring to FIG. 5, there is shown an ultrasound diagnosis image displayed in accordance with the operation being set in the first preferred embodiment in accordance with the invention.

In case of obtaining the ultrasound diagnosis image containing a fetus image, as shown in FIG. 5, some operation such as displaying of B mode image B1 will be executed so as to comply with the parameters extracted by inputting the coordinate positions corresponding to the lower abdominal region containing the fetus in the second body mark BM2. For example, in this embodiment, the parameters such as frequency, frame averaging, gray scale map, width, depth, and focus for the image quality setting will be automatically set, then the following operation sequence will be automatically executed so as to comply with the parameters automatically set. Thereafter the imaged B mode image B1 will be displayed.

As can be appreciated from the foregoing description, in this preferred embodiment, a body mark indicative of the shape of the imaging region of the examinee is first displayed on the display screen, then the coordinate positions of the body mark displayed on the display screen is input. Based on the coordinate positions of the body mark having been input, the operation of the ultrasound diagnosis apparatus 1 will be automatically controlled. In this embodiment, some parameters corresponding to the input coordinate positions of the body mark is extracted from the database in which each of the coordinate positions of the body mark is mutually associated with each of the parameters for setting the operation of the ultrasound diagnosis apparatus, then the operation of the ultrasound diagnosis apparatus will be controlled so as to comply with the extracted parameters. For example, setting parameters will be automatically set by using a database in which the setting parameters for setting the image quality of the ultrasound diagnosis image as parameters are associated with the coordinate positions of the body mark. In accordance with this preferred embodiment, since such setting as image quality setting is automatically set by selecting the coordinate positions of the body mark, then the operation is executed by automatic control, the operation of the apparatus will be facilitated, allowing the improvement of the operation efficiency.

### Second Embodiment

Now the second preferred embodiment in accordance with the invention will be described in greater details herein below.

In this preferred embodiment, the operation sequence at the time when the ultrasound diagnosis apparatus operates by complying with the automatically set parameters (ST13), after the coordinate positions of the body mark are input (ST12), is different from the first preferred embodiment. This preferred embodiment is identical to the first preferred embodiment above except for this point. Therefore the description of the similar contents will be omitted.

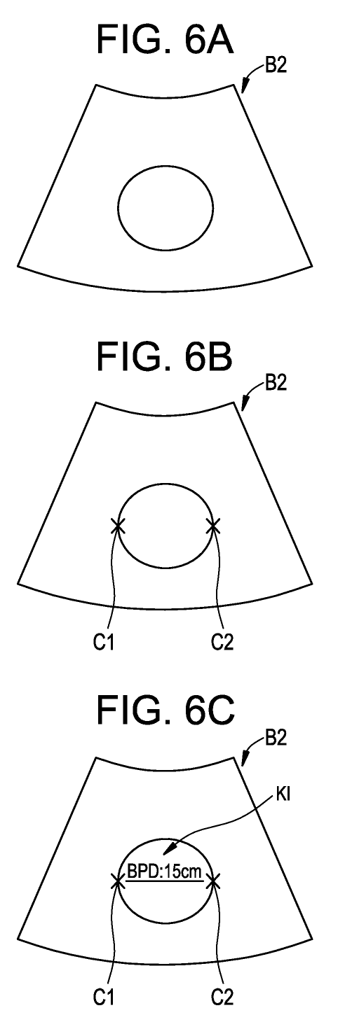

Now referring to FIG. 6 there is shown an ultrasound diagnosis image displayed when the measuring setting in the second preferred embodiment in accordance with the invention is performed.

As similar to the first preferred embodiment, after performing the input of the coordinate positions of the body mark (ST12), as shown in FIG. 6 (a), a B mode image B2 for the head of the fetus is obtained. Thereafter, in the B mode image B2, the control unit 324 will automatically set the biparietal diameter (BTP) of the head of the fetus as the measuring item to perform the measurement. In this preferred embodiment the database in which the measurement setting parameters for setting the measurement on the ultrasound diagnosis image are associated with the coordinate positions of the body mark is used to automatically set the measurement setting parameters. Alternatively, the database in which the comment setting parameters for setting the comment on the ultrasound diagnosis image are associated with the coordinate positions of the body mark may be used for automatically setting the comment setting parameters.

Then, for example, first measuring point C1 and second measuring point C2 will be displayed on the display screen of the display unit 41 by the control unit 324. Thereafter, as shown in FIG. 6 (b), the operator uses an input device such as the track ball to define the positions of the first measuring point C1 and the second measuring point C2 on the B mode image B2. The region defined by the first measuring point C1 and the second measuring point C2 will be automatically set as the measuring region by the control unit 324, then the distance between the points defined will be measured and the measurement results will be automatically obtained. As shown in FIG. 6 (c), the measurement result image KI indicative of the result of the measurement will be displayed on the display screen of the display unit 41 by the control unit 324.

As can be appreciated from the foregoing description, in this preferred embodiment, as similar to the first preferred embodiment described above, after displaying on the display screen the body mark indicative of the shape of the imaging region of the examinee, the coordinate positions of the body mark displayed on the display screen are to be input. Based on the input coordinate positions the operation of the ultrasound diagnosis apparatus 1 will be automatically controlled. Therefore in this preferred embodiment as similar to the first preferred embodiment described above, since such setting as measurement setting will be automatically set by selecting the coordinate positions of the body mark, then the operation will be automatically controlled, the operation will be facilitated, allowing the improvement of the operating efficiency.

In the implementation of the preferred embodiments in accordance with the invention, the invention is not to be limited to the preferred embodiments described above, but any variation thereof may be adopted.

For example, although the operation of setting such as image quality setting, measurement setting and comment setting when imaging a fetus is described, the invention is not to be limited thereto. For example, the invention is applicable to the cases in which a body mark is displayed for any other regions including the abdomen and heart, and any setting is done so as to correspond to the coordinate positions specified in the body mark.

In addition, in the preferred embodiments described above, although the BPD has been described as the measuring item, the invention is not limited thereto. For example, the invention is also applicable to the case in which, as the available measuring items such as parameters for the crown rump length (CRL), the fetal femur length (FFL), the fetal thorax diameter (FTD), the left ventricular internal diameter diastole (LVIDd), the interventricular septal thickness diastole (IVSd), the left ventricular posterior wall thickness diastole (LVPWd) may be set, and the apparatus operates so as to comply with the parameters set. As the comment setting, the invention is also applicable to the case in which the available comment setting such as Aolic Arch, Axill V, PV, TV, Apcial, kidneys, Female, Stomach, Aonta may be set and the apparatus operates so as to comply with the parameters set.

Many widely different embodiments of the invention may be configured without departing from the spirit and the scope of the present invention. It should be understood that the present invention is not limited to the specific embodiments described in the specification, except as defined in the appended claims.

1. An ultrasound diagnosis apparatus for generating an ultrasound diagnosis image of an imaging region based on an echo signal obtained by performing a scan of transmitting an ultrasound beam to the imaging region of an examinee and by receiving an ultrasound echo reflected in the imaging region, the ultrasound diagnosis apparatus comprising:
a display unit for displaying a body mark indicating the shape of the imaging region on a display screen;
an input unit for inputting the coordinate positions of the body mark displayed on the display screen by the display unit; and
a controller unit for controlling the operation of the ultrasound diagnosis apparatus based on the coordinate positions of the body mark to be input by the input unit.

2. An ultrasound diagnosis apparatus according to claim 1, further comprising:
a storage unit for storing a database in which each of the coordinate positions of the body mark is mutually associated with each of parameters for setting the operation of the ultrasound diagnosis apparatus, wherein:
the controlling unit extracts, based on the coordinate positions of the body mark being input by the input unit, the parameters corresponding to the coordinate positions from the database stored in the storage unit, to control the ultrasound diagnosis apparatus so as to comply with thus extracted parameters.

3. An ultrasound diagnosis apparatus according to claim 2, wherein:
the storage unit stores a database in which the image quality setting parameters for setting the image quality of the ultrasound diagnosis image as the parameters are associated with the coordinate positions of the body mark.

4. An ultrasound diagnosis apparatus according to claim 3, wherein:
the image quality setting parameters in the database include at least any two of frequency, frame averaging, gray scale map, width, depth, and focus.

5. An ultrasound diagnosis apparatus according to claim 2, wherein:
the storage unit stores a database in which the measurement setting parameters for setting the measurement of the ultrasound diagnosis image as the parameters are associated with the coordinate positions of the body mark.

6. An ultrasound diagnosis apparatus according to claim 3, wherein:
the storage unit stores a database in which the measurement setting parameters for setting the measurement of the ultrasound diagnosis image as the parameters are associated with the coordinate positions of the body mark.

7. An ultrasound diagnosis apparatus according to claim 5, wherein:
the measurement setting parameters in the database include at least any two of the crown rump length (CRL), the fetal femur length (FFL), the fetal thorax diameter (FTD), the left ventricular internal diameter diastole (LVIDd), the interventricular septal thickness diastole (IVSd) and the left ventricular posterior wall thickness diastole (LVPWd).

8. An ultrasound diagnosis apparatus according to claim 2, wherein:
the storage unit stores a database in which the comment setting parameters for setting the comment of the ultrasound diagnosis image as the parameters are associated with the coordinate positions of the body mark.

9. An ultrasound diagnosis apparatus according to claim 3, wherein:
the storage unit stores a database in which the comment setting parameters for setting the comment of the ultrasound diagnosis image as the parameters are associated with the coordinate positions of the body mark.

10. An ultrasound diagnosis apparatus according to claim 5, wherein:
the storage unit stores a database in which the comment setting parameters for setting the comment of the ultrasound diagnosis image as the parameters are associated with the coordinate positions of the body mark.

11. A controlling method of an ultrasound diagnosis apparatus for generating an ultrasound diagnosis image of an imaging region based on an echo signal obtained by performing a scan of transmitting an ultrasound beam to the imaging region of an examinee and receiving an ultrasound echo reflected in the imaging region, the method comprising the steps of:
displaying on a display screen a body mark indicative of the shape of the imaging region;
inputting the coordinate positions of the body mark displayed on the display screen by the displaying step; and
controlling the operation of the ultrasound diagnosis apparatus based on the coordinate positions of the body mark input by the input step.

12. A controlling method of an ultrasound diagnosis apparatus according to claim 11, wherein:
in the controlling step, based on the coordinate positions of the body mark being input in the input step, from the database in which each of coordinate positions of the body mark is mutually associated with each of parameters for setting the operation of the ultrasound diagnosis apparatus, the parameters are extracted so as to comply with the coordinate positions to control the operation of the ultrasound diagnosis apparatus so as to comply with thus extracted parameters.

13. A controlling method of an ultrasound diagnosis apparatus according to claim 12, wherein:
in the controlling step, a database is used in which the image setting parameters for setting the image quality of the ultrasound diagnosis image as the parameters are associated with the coordinate positions of the body mark.

14. A controlling method of an ultrasound diagnosis apparatus according to claim 13, wherein:
the image quality setting parameters in the database include at least any two of frequency, frame averaging, gray scale map, width, depth, and focus.

15. A controlling method of an ultrasound diagnosis apparatus according to claim 12, wherein:
in the controlling step, a database is used in which the measurement setting parameters for setting the measurement of the ultrasound diagnosis image as the parameters are associated with the coordinate positions of the body mark.

16. A controlling method of an ultrasound diagnosis apparatus according to claim 13, wherein:
in the controlling step, a database is used in which the measurement setting parameters for setting the measurement of the ultrasound diagnosis image as the parameters are associated with the coordinate positions of the body mark.

17. A controlling method of an ultrasound diagnosis apparatus according to claim 15, wherein:
the measurement setting parameters in the database include at least any two of the crown rump length (CRL), the fetal femur length (FFL), the fetal thorax diameter (FTD), the left ventricular internal diameter diastole (LVIDd), the interventricular septal thickness diastole (IVSd) and the left ventricular posterior wall thickness diastole (LVPWd).

18. A controlling method of an ultrasound diagnosis apparatus according to claim 12, wherein:
in the controlling step, a database is used in which the comment setting parameters for setting the comment in the ultrasound diagnosis image as the parameters are associated with the coordinate positions of the body mark.

19. A controlling method of an ultrasound diagnosis apparatus according to claim 13, wherein:
in the controlling step, a database is used in which the comment setting parameters for setting the comment in the ultrasound diagnosis image as the parameters are associated with the coordinate positions of the body mark.

20. A controlling method of an ultrasound diagnosis apparatus according to claim 15, wherein:
in the controlling step, a database is used in which the comment setting parameters for setting the comment in the ultrasound diagnosis image as the parameters are associated with the coordinate positions of the body mark.
